(19) United States
(12) Patent Application Publication
(10) Pub. No.: US 2007/0185522 A1
(43) Pub. Date: Aug. 9, 2007

(54) Dilator

(76) Inventors: Gareth Davies, Toronto (CA); Hartley Amanda, Brampton (CA)

Correspondence Address: Louis Tessier, P.O. Box 54029, Town of Mount Royal, QC, H3P 3H4, CA

(21) Appl. No.: 11/727,382

(22) Filed: Mar. 26, 2007

Related U.S. Application Data
(63) Continuation-in-part of application No. 11/265,304, filed on Nov. 3, 2005.
Application No. 11/265,304 is a continuation-in-part of application No. 10/666,301, filed on Sep. 19, 2003, now Pat. No. 7,048,733.
Application No. 11/265,304 is a continuation-in-part of application No. 10/760,479, filed on Jan. 21, 2004.
Application No. 11/265,304 is a continuation-in-part of application No. 10/666,288, filed on Sep. 19, 2003.
Application No. 10/666,288 is a continuation-in-part of application No. 10/347,366, filed on Jan. 21, 2003, now Pat. No. 7,112,197.
(60) Provisional application No. 60/743,722, filed on Mar. 24, 2006.

(51) Int. Cl. A61M 29/00

(52) U.S. Cl. 606/191

(57) ABSTRACT

A dilator, the dilator being positionable substantially adjacent an aperture in a tissue and usable to enlarge the aperture, the dilator comprising a substantially elongated member, the substantially elongated member defining a member proximal end section, a substantially longitudinally opposed member distal end section and a member middle section extending therebetween, the member middle section including a tube defining a tube lumen extending substantially longitudinally therethrough and a reinforcing component located, at least in part, within the tube lumen, the member middle section being substantially less mechanically deformable than the member distal end section; whereby having the member distal end section substantially more mechanically deformable than the member middle section reduces risks of injuring the tissue with the member distal end section when positioning the member distal end section substantially adjacent the aperture while allowing for the transmission of substantially longitudinal forces from the member proximal end section to the member distal end section to enlarge the aperture by pushing, at least in part, the member distal end section through the aperture.

### REFERENCES TO PARENT AND CO-PENDING APPLICATIONS

This application is a continuation-in-part of co-pending U.S. application Ser. No. 11/265,304, filed Nov. 3, 2005. Ser. No. 11/265,304 is a continuation-in-part of U.S. application Ser. No. 10/666,301, filed Sep. 19, 2003 (now U.S. Pat. No. 7,048,733, issued on May 23^{rd}, 2006) and a continuation-in-part of co-pending U.S. application Ser. No. 10/760,479, filed Jan. 21, 2004 and a continuation-in-part of co-pending U.S. application Ser. No. 10/666,288, filed Sep. 19, 2003, which is a continuation-in-part of U.S. application Ser. No. 10/347,366, filed Jan. 21, 2003 (now U.S. Pat. No. 7,112,197, issued on Sep. 26^{th}, 2006). This application also claims priority from and the benefit of U.S. provisional patent application Ser. No. 60/743,722, filed Mar. 24, 2006. All of these US Patents and Patent Applications are hereby incorporated by reference in their entirety.

### FIELD OF THE INVENTION

The present invention relates generally to methods and devices usable to enlarge apertures. More specifically, the present invention is concerned with a dilator.

### BACKGROUND OF THE ART

Several surgical procedures exist wherein a device comprising an elongated member defining a lumen may be inserted into a patient's body. For example, during minimally invasive heart surgery, such a device may be introduced into the femoral vein and directed towards the heart. In some instances, force may be applied to the proximal region of the device, such that the force may be transmitted to the distal region of the device and perform a function within the body, for example dilating an aperture. In some instances, depending on the nature of the function being performed, the device may not provide enough stiffness or column strength to transmit the force. However, stiffening the whole device increases the risks of injuring tissues located adjacent the region to which the force applied to the proximal region of the device is to be transmitted (i.e. adjacent to the distal end section of the device), or tissues located in the path through which the device is introduced in the patient's body.

Against this background, there exists a need in the industry to provide novel dilators. An object of the present invention is therefore to provide such a dilator.

### SUMMARY OF THE INVENTION

In a broad aspect, the invention provides a dilator, the dilator being positionable substantially adjacent an aperture in a tissue and usable to enlarge the aperture, the dilator comprising:
a substantially elongated member, the substantially elongated member defining a member proximal end section, a substantially longitudinally opposed member distal end section and a member middle section extending therebetween, the member middle section including a tube defining a tube lumen extending substantially longitudinally therethrough and a reinforcing component located, at least in part, within the tube lumen, the member middle section being substantially less mechanically deformable than the member distal end section;

whereby having the member distal end section substantially more mechanically deformable than the member middle section reduces risks of injuring the tissue with the member distal end section when positioning the member distal end section substantially adjacent the aperture while allowing for the transmission of substantially longitudinal forces from the member proximal end section to the member distal end section to enlarge the aperture by pushing, at least in part, the member distal end section through the aperture.

Advantageously, the dilator reduces risks of injuring the tissue with the member distal end section when positioning the member distal end section substantially adjacent the aperture, while allowing for the transmission of longitudinal forces from the member proximal end section to the member distal end section to enlarge the aperture by pushing, at least in part, the member distal end section through the aperture.

The inventors found the new and unexpected result that such a dilator, with suitable stiffness to transmit longitudinal force, may be manufactured to have dimensions allowing for use with standard electrosurgical devices within the body.

In another broad aspect, the invention provides a dilator, the dilator being positionable substantially adjacent an aperture in a tissue and usable to enlarge the aperture, the dilator comprising:
a substantially tubular component, the substantially tubular component having a substantially elongated configuration and defining a component proximal end section, a substantially longitudinally opposed component distal end section and a component middle section extending therebetween, the component middle section being substantially less mechanically deformable than the component distal end section;
whereby having the component distal end section substantially more mechanically deformable than the component middle section reduces risks of injuring the tissue with the component distal end section when positioning the component distal end section substantially adjacent the aperture while allowing for the transmission of substantially longitudinal forces from the component proximal end section to the component distal end section to enlarge the aperture by pushing, at least in part, the component distal end section through the aperture.

Other objects, advantages and features of the present invention will become more apparent upon reading of the following non-restrictive description of certain embodiments thereof, given by way of example only with reference to the accompanying drawings.

### BRIEF DESCRIPTION OF THE DRAWINGS

In order that the invention may be readily understood, embodiments of the invention are illustrated by way of examples in the accompanying drawings, in which:

FIG. 1 is a side view of a device in accordance with one embodiment of the present invention;

FIGS. 2A-2D are perspective views of various embodiments of the distal end section of a device in accordance with the present invention;

FIG. 3 is a perspective view of one embodiment of a reinforcing component in accordance with the present invention;

FIG. 4 is a cross-sectional view of a device in accordance with one embodiment of the present invention;

FIG. 5 is a side view of one embodiment of a system in accordance with the present invention;

FIG. 6A is a schematic view of an embodiment of a device in accordance with the present invention with the device in an undeformed configuration; and

FIG. 6B is a schematic view of the device shown in FIG. 6A with the device in a deformed configuration.

### DETAILED DESCRIPTION

With specific reference now to the drawings in detail, it is stressed that the particulars shown are by way of example and for purposes of illustrative discussion of certain embodiments of the present invention only. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Device

In one broad aspect, embodiments of the present invention relate to a device having sufficient stiffness to transmit longitudinal forces, applied to a proximal end section of the device, to the distal end section of the device in order to perform a function within a patient's body. As one feature of this broad aspect, embodiments of the present invention may comprise a device for dilating or enlarging a hole, aperture, or perforation in a material or tissue within the patient of a body. Such a device is often referred to as a dilator. As another feature of this aspect, embodiments of the present invention may comprise a device for guiding, positioning, holding, and/or supporting a device, for example an electrosurgical device, within the body.

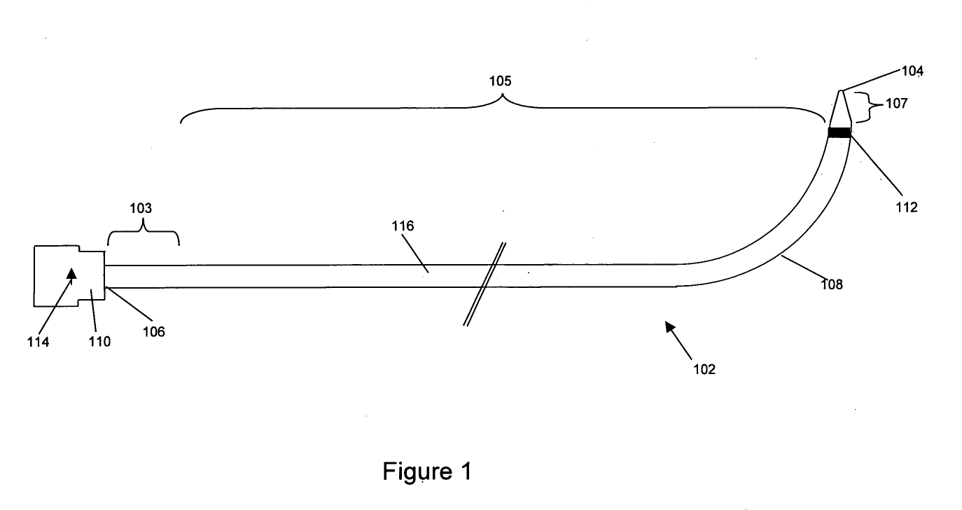

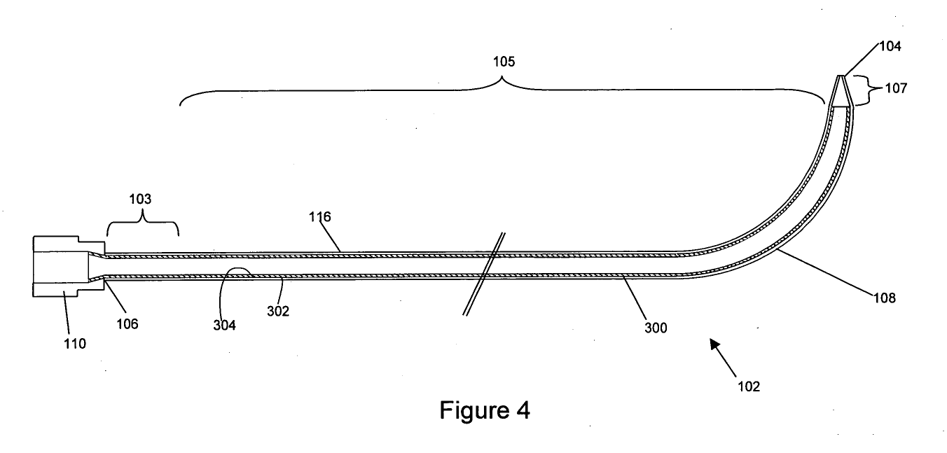

As shown in the embodiment of FIG. 1, a device of the present invention may comprise a substantially elongated member 102 having member proximal end section 103 ending in open proximal end 106, a substantially longitudinally opposed member distal end section 107 ending in open distal end 104, a member middle section 105 extending longitudinally therebetween, and a lumen extending between proximal end 104 and distal end 106. Elongated member 102 includes a tube 116 in the member middle section 105, which defines a tube lumen (described further herein below with reference to FIG. 4). The member proximal end section 103 and the member distal end section 107 extend from the tube 116. In some such embodiments, member proximal end section 103 and member distal end section 107 may extend integrally from tube 116. In other embodiments, member proximal end section 103 and member distal end section 107 may comprise additional components, each defining a lumen that is in communication with the tube lumen. Thus, the elongated member lumen comprises the tube lumen, plus the lumens defined by member proximal end section 103 and member distal end section 107.

In some embodiments, the transverse cross-sectional shape of elongated member 102 may be substantially circular. In other embodiments, other shapes are possible, such as ovoid, for example, and the invention is not limited in this regard. The cross-sectional diameter may vary along the length of elongated member 102, as will be discussed hereinbelow.

The size of elongated member 102 may vary according to the particular application. For example, in applications wherein elongated member is used to guide a catheter or other device from a femoral vein to a patient's heart, the length of elongated member 102 may be between about 60 cm and 75 cm, more specifically about 67 cm. In other embodiments, elongated member 102 may be between about 50 cm and 100 cm in length. In yet further embodiments, other lengths may be possible depending on the specific application.

In some embodiments, wherein the outer diameter of elongated member 102 is constant over the length of elongated member 102, the outer diameter of elongated member 102 may be between about 0.15 cm and about 0.37 cm. In other embodiments, the outer diameter of elongated member 102 may taper or decrease towards distal end 104. For example, the outer diameter of the proximal end section 103 of elongated member 102 may be between about 0.15 cm and about 0.37 cm, and in some embodiments may be about 0.25 cm; and the outer diameter of distal end section 107 may be between about 0.025 cm and 0.25 cm. In other words, the distal end section 107 of the elongated member 102 tapers in a direction leading away from the middle section 105 of the elongated member 102. The point at which the diameter begins to taper may vary depending on the application. In some embodiments, the diameter may begin to taper between about 5 and about 15 mm from distal end 104. The change in diameter may be substantially abrupt, occurring over a length of between about 5 mm and about 8 mm, or may be substantially smooth, occurring over a length of between about 10 mm and about 15 mm. Furthermore, the device is not limited to a single region of decreasing diameter, and may comprise several changes of diameter over the length of elongated member 102.

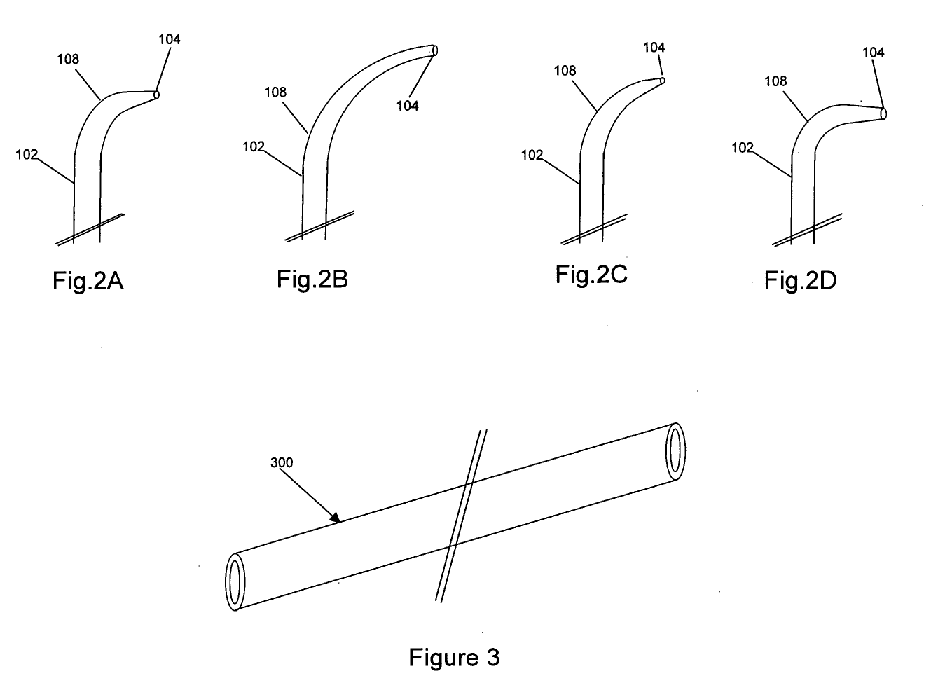

In some embodiments, elongated member 102 may be substantially straight. In other embodiments, as shown in FIGS. 2A-2D, elongated member 102 may be curved. The curve 108 may be of a number of angles, and may begin at various positions. In some embodiments, elongated member 102 may begin to curve between about 30 mm and about 100 mm from distal end 104, and the curve may traverse up to about 89° of a circle. For example, in one embodiment, elongated member 102 may begin to curve about 75 mm from distal end 104, and the curve may traverse about 45° of a circle. In another example, curve 108 may begin about 50 mm from distal end 104, and may traverse about 89° of a circle. In other examples, the curve 108 may traverse from between about 30° to about 89° of a circle. Furthermore, curve 108 may be substantially abrupt, occurring over a short length, or may be substantially gradual, occurring over a longer length. In addition, curve 108 may not extend all the way to distal end 104, and therefore a portion of elongated member 102 adjacent to or at distal end 104 may be substantially straight, as shown in FIG. 2D. In some embodiments, this straight portion may be about 5 to about 15 mm, for example the distalmost 10 mm of elongated member 102 may be substantially straight. In further embodiments, elongated member 102 may comprise more than one curve in more than one plane.

Curve 108 may be applied to elongated member 102 by a number of means. For example, a heated mold may be used to apply curve 108 to elongated member 102 during manufacturing. That is, elongated member 102 may be manufactured in a straight conformation, and then curve 108 may be applied using a heated mold. In another example, elongated member 102 may be initially manufactured with curve 108 already incorporated.

Elongated member 102 may be manufactured from a number of materials. For example, tube 116 of elongated member 102 may be made out of a material including, but not limited to, polymers, for example polyurethane, polyvinylchloride, polypropylene, PEEK™ (oxy-1,4-phenyleneoxy-1,4-phenylene-carbonyl-1,4-phenylene), PEBAX™ (polyamide polyether block copolymer), polyetheretherketone, polyethylene block amide, and any suitable medical grade rubbers or plastics or any combination thereof. Furthermore, different portions of elongated member 102 may be made from different materials. For example, the tube 116 may be made from a material that may provide stiffness or column strength; however distal end section 107 may be made from a material that may provide more flexibility. In addition, some embodiments of elongated member 102 may comprise various coatings. For example, elongated member 102 may be coated with a silicone or parylene coating to provide lubricity. In other embodiments, elongated member 102 may be coated with an antithrombotic coating. Other coatings may also be used and the invention is not limited in this regard.

Elongated member 102 may be manufactured by a variety of different methods. For example, tube 116 may be made by extrusion, co-extrusion, injection moulding or other tube-forming methods. The taper may be applied to elongated member 102 by heated die molding, machining or necking.

In some embodiments, a hub 110 may be operatively connected to proximal end 106 of elongated member 102. Hub 110 may function to secure or lock elongated member 102 to other devices, such as cannulae, probes, or catheters, for example. Hub 110 may define a lumen extending therethrough in communication with the lumen defined by elongated member 102. Hub 110 may be a luer lock, a push-button connector, or any other coupling means that would not interfere with the functioning of the device. Hub 110 may be attached to elongated member 102 via a number of methods, such as the use of adhesives, friction fitting, crimping or by a screw-mechanism, for example. Furthermore, hub 110 may be detachable from elongated member 102, for example by un-screwing or by pressing or releasing a button and/or switch.

In some embodiments, elongated member 102 may comprise a reinforcing component 300, seen in FIG. 3, which may take the form of a cylinder or a cannula. As shown in FIG. 4, reinforcing component 300 is disposed at member middle section 105 within the tube lumen defined by tube 116, and may also extend into other portions of elongated member 102, for example into the lumen of proximal end section 103. In some embodiments, reinforcing component 300 may function to effectively increase the stiffness or column strength of the member middle section 105 and may be located substantially coaxially relatively thereto. For example, in some embodiments, reinforcing component 300 may have a flexural rigidity of at least 0.05 N/m². In some embodiments, reinforcing component 300, when fully disposed within elongated member 102, extends through middle section 105. In further embodiments, reinforcing component 300 may extend through middle section 105 and all or a portion of proximal end section 103. In one particular embodiment, reinforcing component 300 may extend proximally from about the point in elongated member 102 at which elongated member 102 begins to taper. In yet further embodiments, reinforcing component 300 may extend proximally beyond proximal end 106 of elongated member 102, for example into hub 110. In further embodiments, reinforcing component 300 may extend between other points of elongated member 102, and the invention is not limited in this regard.

In some embodiments, the inner diameter of reinforcing component 300 may be between about 0.08 cm and about 0.18 cm; and the outer diameter of reinforcing component 300 may be between about 0.12 cm and about 0.01 cm. For example, in one embodiment, the inner diameter of reinforcing component 300 may be about 0.14 cm, and the outer diameter of reinforcing component 300 may be about 0.16 cm; the wall thickness of reinforcing component 300 thereby being about 0.02 cm. Furthermore, in some embodiments, as shown for example in FIG. 4, the proximal region of reinforcing component 300 may be slightly flared. For example, the majority of reinforcing component 300 may have an outer diameter of about 0.10 cm to about 0.15 cm, and more specifically, about 0.14 cm; however, about 0.64 cm to about 1.9 cm from the proximal end of reinforcing component 300, the outer diameter may increase to between about 0.17 cm and 0.50 cm, for example to about 0.25 cm. This flare may function to substantially anchor reinforcing component 300 within elongated member 102 or hub 110.

Reinforcing component 300 may be manufactured from a number of different materials including, but not limited to, stainless steels, titanium alloys, nickel alloys, thermoplastics, filled thermoplastics and any combinations thereof. In one particular embodiment, reinforcing component 300 is manufactured from annealed stainless steel. In addition, reinforcing component 300 may be radiopaque, or may comprise radiopaque markings. Furthermore, different portions of reinforcing component 300 may be made from different materials, and the invention is not limited in this regard. For example, the majority of reinforcing component 300 may be made from stainless steel; however a curved portion may be made from a more flexible material, such as nitinol. In another embodiment, a curved portion of the reinforcing component may have notches cut out of it in order to alter the flexibility thereof.

Reinforcing component 300 may be attached, bonded, or otherwise operatively connected to tube 116 by a number of means. For example, in one embodiment, an adhesive may be applied to the proximal region of reinforcing component 300, and reinforcing component 300 may then be inserted into tube 116 such that portions of the proximal regions of reinforcing component 300 and tube 116 adhere together. In some embodiments, prior to the application of an adhesive, the outer surface of the proximal region of reinforcing component 300 may be roughened, for example by grit-blasting. This may aid in bonding reinforcing component 300 to tube 116. In other embodiments, tube 116 may be formed around reinforcing component 300 during manufacturing. For example, reinforcing component 300 may be dipped into a molten material that may coat reinforcing component 300 to form tube 116.

In some embodiments, as described hereinabove, elongated member 102 may be curved. In these embodiments, reinforcing component 300 may also be curved. In one embodiment, a curve may be applied to reinforcing component 300 prior to insertion into tube 116. For example, reinforcing component 300 may be heated using a heated mold, and then curved manually such that, when reinforcing component 300 cools, it may maintain its curved shape. Reinforcing component 300 may then be inserted into tube 116, and tube 116 may be sufficiently flexible so as to assume the curved shape of reinforcing component 300. In other embodiments, reinforcing component 300 may be inserted into tube 116 in a substantially straight conformation. A curve may then be applied to both reinforcing component 300 and tube 116 substantially concurrently, for example by heating and then manually applying a curve to the device.

In some embodiments, elongated member 102 may comprise means to aid the user in determining the position of the device within the body. For example, one or more radiopaque markings 112 may be coupled to elongated member 102, for example, and non-limitingly, located substantially adjacent the distal end 104, to allow for better visualization of elongated member 102 under fluoroscopic imaging. Radiopaque marking(s) 112 may be in the form of a metal band, for example a platinum or iridium band, or may be in the form of a plastic that has been filled with a radiopaque material, such as bismuth, for example. In other embodiments, elongated member 102 may comprise one or more visual or tactile markings, for example depth markings, in order to allow the user to establish how far the device has been inserted into the body. Such markings may be in the form of a colored band, notch, or dot, for example, or a raised bump or protrusion. In further embodiments, proximal end section 103 of elongated member 102 and/or the hub 110 may comprise at least one marking 114 to indicate a direction of a curve in the distal end section 107 of elongated member 102, when such a curve is present. Such a marking may be in the form of a visual marking such as an arrow, for example and/or a tactile marking, such as a raised surface.

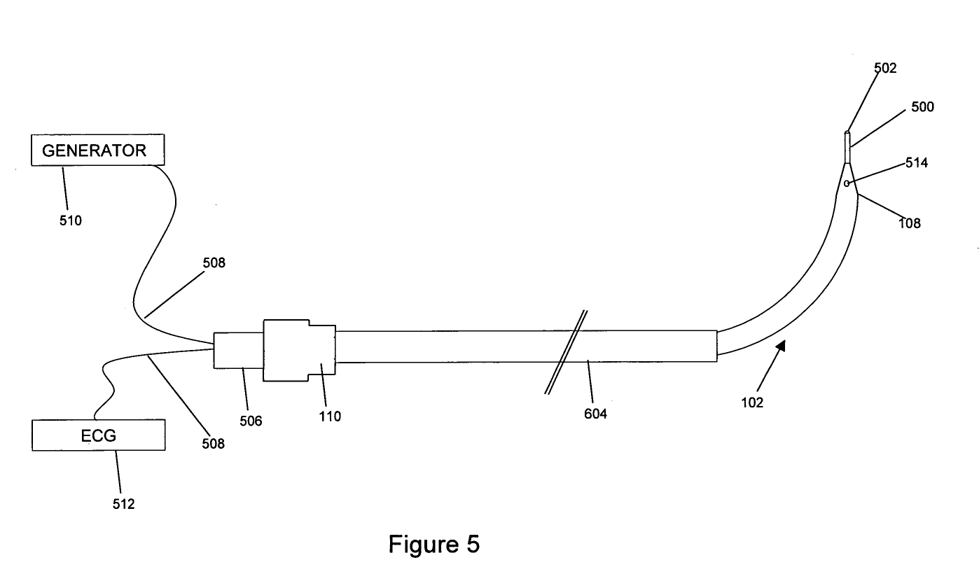

In some embodiments, and with reference now to FIG. 5, a wall of elongated member 102 may define at least one opening or lateral aperture 514 extending substantially radially outwardly from a lumen of the elongated member 102, to allow the lumen of elongated member 102 to be in communication with the outside environment. The aperture(s) may be positioned anywhere along elongated member 102, for example at the distal end section 107 of elongated member 102. The aperture(s) may be of any number and size that does not interfere with the functioning of the device; for example, elongated member 102 may comprise 2 apertures each with a diameter of between about 1 mm and about 1.5 mm. Aperture(s) may be used to deliver a fluid, such as a contrast agent, for example, through elongated member 102 to the outside environment. In embodiments wherein elongated member 102 comprises a reinforcing component 300 disposed within the tube lumen at middle section 105, the aperture(s) may be defined in distal end section 107 of elongated member 102, in order to avoid reinforcing component 300. Alternatively, tube 116 may define one or more apertures in a wall therethrough, and reinforcing component 300 may also define one or more apertures in a wall therethrough, such that the apertures of tube 116 are aligned with the apertures in reinforcing component 300.

Systems of the present invention may comprise several auxiliary devices in the addition to the device described above. For example, as shown in the embodiment of FIG. 5, elongated member 102 may be structured to allow a device, for example an electrosurgical device 500, to be disposed therethrough. Electrosurgical device 500 may be, for example, a wire or catheter structured to deliver energy, for example radiofrequency current, from its distal tip 502. Elongated member 102 may itself be sized to fit within a sheath 504, which may assist in guiding elongated member 102 to a target site within the body. Alternatively, elongated member 102 may assist in guiding sheath 504 to a target site within the body. In use, hub 110 may be operatively connected to a second hub 506, which may be operatively connected to one or more connector cables 508 and to electrosurgical probe 500. In the embodiment shown, one connector cable 508 may operatively connect electrosurgical probe 500 to a source of energy, such as generator 510, while another connector cable 508 may operatively connect electrosurgical probe 500 to a monitoring system, such as ECG monitor 512. In other embodiments, further auxiliary devices may be included. For example, systems of the present invention may comprise visualization devices such as endoscopes, devices for measurement of blood pressure, or cooling devices. Any of the aforementioned devices may be packaged together in a kit.

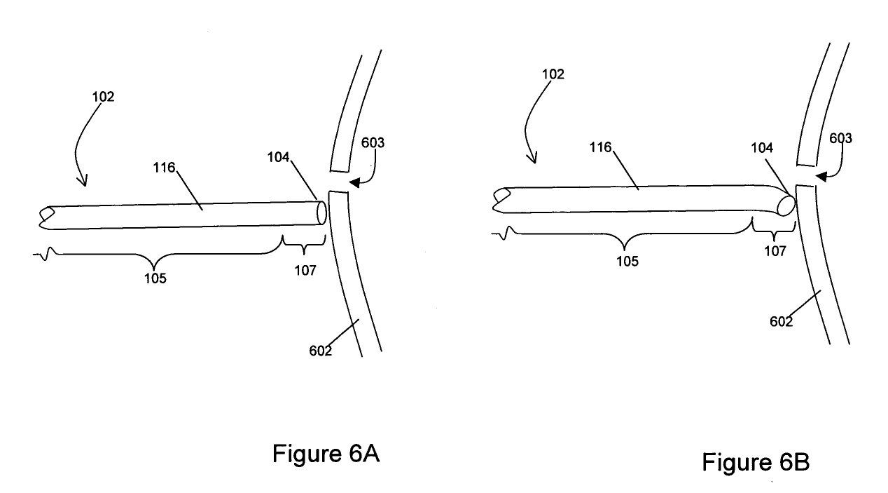

Referring to FIGS. 6A and 6B, there is shown an embodiment of elongated member 102. As will be described further herein below, FIG. 6A shows elongated member 102 in an undeformed configuration, while FIG. 6B shows elongated member 102 in a deformed configuration. As detailed hereinbelow, the elongated member 102 is positionable substantially adjacent an aperture in a tissue and usable to enlarge the aperture. As described herein above, the elongated member 102 defines a member proximal end section 103, a substantially longitudinally opposed member distal end section 107 and a member middle section 105 extending therebetween. Furthermore, as mentioned hereinabove, in some embodiments of the invention, the elongated member middle section 105 includes a tube 116 defining a tube lumen extending substantially longitudinally therethrough and a reinforcing component 300 located, at least in part, within the tube lumen. In some embodiments of the invention, the member middle section 105 is substantially less mechanically deformable than the member distal end section 107, due to the presence of reinforcing component 300 at member middle section 105. Having the member distal end section 107 substantially more mechanically deformable than the member middle section 105 reduces risks of injuring a tissue, schematically represented as element 602 in FIGS. 6A and 6B, with the member distal end section 107 while the member distal end section 107 is being positioned substantially adjacent an aperture 603 in tissue 602, while allowing for the transmission of longitudinal forces from the member proximal end section 103 to the member distal end section 107 to enlarge the aperture 603, by pushing, at least in part, the member distal end section 107 through the aperture 603. In other words, when the member distal end section 107 is in the process of being positioned substantially adjacent aperture 603, but is contacting a different portion of tissue 602, and substantially longitudinal force is applied at member proximal end section 103, member distal end section 107 will deform sufficiently so that the risk of injuring tissue 602 is reduced. This is made possible by a relatively large deformability of the member distal end section 107 as compared to the deformability of the member middle section 105 upon the exertion of a substantially longitudinal force, as seen in FIG. 6B. For example, in some embodiments, a flexural rigidity of the member middle section 105 is at least about 30 times larger than a flexural rigidity of the member distal end section 107. However, when the member distal end section 107 is finally positioned substantially adjacent to aperture 603 and substantially longitudinal force is applied at member proximal end section 103, member distal end section 107 may advance through aperture 603 in order to enlarge aperture 603.

In some embodiments of the invention, a ratio between a length of the member distal end section 107 and a length of the member middle section 105 is from about 1:200 to about 1:33. In some embodiments of the invention, the reinforcing component 300 is made from a metal and the member distal end section 107 is substantially electrically insulating. This allows the use of elongated member 102 with electrosurgical devices because the electrically insulated member distal end section 107 prevents electrical energy from being transmitted from the electrosurgical device to reinforcing component 300 and into the tissue.

As seen in FIG. 4, in some embodiments of the invention, the reinforcing component 300 defines a reinforcing component radially outermost surface 302 and tube 116 defines a tube radially innermost surface 304. The reinforcing component radially outermost surface 302 and the tube radially innermost surface 304 are in contact with each other. For example, the reinforcing component radially outermost surface 302 and the tube radially innermost surface 304 are bonded to each other. Also, to improve bonding between these two surfaces, in some embodiments of the invention, the reinforcing component outermost surface 302 is, at least in part, substantially roughened.

Methods

In one broad aspect, embodiments of a method of the present, invention may involve the application of a force to the proximal end section of a device, wherein the force is transmitted to the distal end section of the device such that it may perform a function within a patient's body. As a feature of this aspect, the function may comprise the dilation or enlargement of a hole or aperture in a tissue or material within the body. In another aspect, embodiments of the method of the present invention may involve the guiding of an electrosurgical device, such as a probe or catheter, to a target site within the body. As a feature of this aspect, some embodiments may further comprise a step of perforating a tissue, for example a tissue of the atrial septum, using radio frequency energy, followed by a step of enlarging the perforation.

In general, some particular embodiments of the method of the present invention may comprise the steps of: guiding a device to a target site within a patient's body, delivering energy to the target site, and applying force to perform a function on the target site. Specific details related to each of these steps will be further discussed hereinbelow.

In one embodiment, the target site, for example the atrial septum of the heart, may be accessed via a femoral vein. In this embodiment, a user may introduce a guidewire, and advance it towards the heart. A guiding sheath, such as sheath 504, may then be introduced into the femoral vein over the guidewire, and advanced towards the patient's heart. The guidewire and sheath may be positioned in the superior vena cava. This step may be performed with the aid of fluoroscopic imaging. When the sheath is positioned, a dilator, such as elongated member 102 described hereinabove, may be introduced into the sheath, and advanced through the sheath into the superior vena cava. The step of advancing the dilator may be performed while an electrosurgical probe, such as probe 502, is disposed within the dilator. In alternate embodiments, the dilator and sheath may be advanced simultaneously into and through the patient's vasculature, with the dilator fully or partially disposed within the sheath. When the guidewire, sheath, and dilator have been positioned in the superior vena cava, they may be withdrawn slightly, such that they enter the right atrium of the heart.

When the distal end section of the dilator has reached the right atrium, the user may proceed to position the distal end section of the dilator against the atrial septum. The user may now introduce an electrosurgical probe into the dilator if it has not already been inserted. The probe may be inserted such that a distal end of the probe may protrude from an open distal end of the dilator. The position of the distal ends of the probe and dilator may now be adjusted to position the distal end of the probe at a target site, for example against the fossa ovalis of the atrial septum. Electrocardiogram (ECG) measurements, fluoroscopic visualization, as well as other techniques, may be used to aid in positioning the distal end of the probe at the appropriate site. When the probe and dilator have been positioned, a variety of optional steps may be performed, such as measuring a property of the target site, or delivering a treatment to the target site. For example, with the probe and dilator positioned at the target site, energy may be delivered from the probe to the target tissue. In some embodiments, the energy may be radiofrequency current, and may be delivered from a generator such as generator 510. The energy may function to vaporize cells in the vicinity of the probe, thereby creating a void or perforation through the target tissue. As energy is being delivered, the user may apply force to the proximal region of the probe to advance the probe into and through the perforation. When the probe has passed through the target tissue, that is, when it has reached the left atrium (in this particular example), energy delivery may be stopped. Further details regarding the radiofrequency perforation of tissue, for example an atrial septum, may be found in U.S. Pat. No. 6,565,562, or U.S. patent application Ser. No. 11/265,304 (filed on Nov. 3^{rd}, 2005), both of which are incorporated herein by reference.

At this point in the procedure, the diameter of the perforation may generally be substantially the same as that of the probe. In some embodiments, the user may wish to enlarge the perforation, such that other devices, for example ablation catheters or other surgical devices, may pass therethrough. To do this, the user may apply force to the proximal end section of the dilator. The force may, in some embodiments, be applied in the cranial or cephalad direction. Due to the flexural rigidity, or stiffness, of the middle section of the dilator, the force may be transmitted to the distal end section of the dilator, and may cause the distal end section of the dilator to enter and dilate, or enlarge, the perforation, and pass through to the left atrium. The probe may aid in guiding the dilator through the perforation, in that it may act as a rail for the dilator to move across. As more force is applied to the proximal region of the dilator, portions of the dilator of larger diameter, as discussed hereinabove with respect to a tapered device, may proceed to enter the perforation, thereby additionally dilating, expanding, or enlarging the perforation. In some embodiments, the user may also apply torque to aid in maneuvering the dilator.

When the perforation has been dilated to a suitable size, which may correspond to the largest diameter of the dilator, the user may stop advancing the dilator. The sheath may then be advanced, for example across the dilator, through the perforation. In some embodiments, the dilator may be retracted prior to advancing the sheath. Alternatively, the sheath may be advanced simultaneously with the dilator. At this point in the procedure, the user may retract the dilator and probe proximally through the sheath, leaving only the sheath in place in the heart. The user may then perform a treatment procedure on the left side of the heart, via the sheath. For example, the user may introduce a device into the femoral vein through the sheath, and may perform a procedure to treat electrical or morphological abnormalities within the left side of the heart.

In other embodiments, rather than the femoral vein, the heart may be accessed via the jugular vein, as is disclosed in U.S. patent application Ser. No. 11/265,304 (filed on Nov. 3^{rd}, 2005), incorporated herein by reference. In this embodiment, a user may introduce a guiding sheath, such as sheath 504, into the heart via the superior vena cava. In such an embodiment, access to the patient's vasculature may be achieved, for example, through a jugular vein, a subclavian vein, or various other points of entry. When the sheath is positioned in the right atrium, a dilator, such as elongated member 102 described hereinabove, may be introduced into the sheath, and advanced through the sheath into the right atrium. When the distal end section of the dilator has reached the right atrium, the user may position the distal end of the dilator against the atrial septum. The user may now introduce an electrosurgical probe into the dilator. When the probe and dilator have been positioned, for example against the fossa ovalis of the atrial septum, energy may be delivered from the probe to the target tissue. The energy may function to vaporize cells in the vicinity of the probe, as described hereinabove. As energy is being delivered, the user may apply force to the proximal region of the probe to advance the probe into the perforation. When the probe has passed through the target tissue, that is, when it has reached the left atrium, energy delivery may be stopped. Force may now be applied to the proximal end section of the dilator. The force may generally be applied in the caudal direction. The force may be transmitted to the distal end section of the dilator, and cause the distal end of the dilator to enter and dilate the perforation, and pass through to the left atrium. The probe may aid in guiding the dilator through the perforation, in that it may act as a rail for the dilator. As more force is applied, portions of the dilator of larger diameter, as discussed hereinabove with respect to a tapered device, may enter the perforation, thereby further dilating the perforation.

In further embodiments, methods of the present invention may be used for surgical procedures involving other regions within the body, and the invention is not limited in this regard. For example, rather than the atrial septum, systems and methods of the present invention may be used to treat pulmonary atresia. In one specific embodiment, as described hereinabove, a sheath, such as sheath 504, may be introduced into the femoral vein of a patient, and guided to the heart. A dilator, such as elongated member 102 described hereinabove, may be introduced into the sheath, and advanced towards the heart, where it may be positioned against the pulmonary valve. A probe may be introduced into the proximal region of the dilator, and guided therethrough, such that it is also positioned against the pulmonary valve. Energy may be delivered from the probe to the pulmonary valve, such that a perforation or void is created therethrough, as described hereinabove. When the probe has passed through the valve, the user may apply a force, generally in the cranial or cephalad direction, to the proximal end section of the dilator. Due to the stiffness and/or flexural rigidity of the middle section of the dilator, the force may be transmitted to the distal end section of the dilator, such that the distal end section of the dilator may enter and dilate the perforation and advance through the pulmonary valve. As regions of the dilator of larger diameter pass through the perforation, the perforation may be further dilated.

Thus, in various embodiments of the method aspect of the present invention, a dilator or other device of the present invention having sufficient stiffness and/or flexural rigidity may be utilized to assist in performing a function at a target site. For example, if the device is used to dilate a perforation, the stiffness and/or flexural rigidity may allow the device to dilate a perforation through a tissue that may otherwise resist dilation, for example a thicker and/or more fibrous tissue.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. For example, in embodiments combining a device, for example a dilator, with another device, for example a sheath, the devices may be packaged together in a kit or may be packaged and/or sold separately.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

We claim:

1. A dilator, said dilator being positionable substantially adjacent an aperture in a tissue and usable to enlarge said aperture, said dilator comprising
a substantially elongated member, said substantially elongated member defining a member proximal end section, a substantially longitudinally opposed member distal end section and a member middle section extending therebetween, said member middle section including a tube defining a tube lumen extending substantially longitudinally therethrough and a reinforcing component located, at least in part, within said tube lumen, said member middle section being substantially less mechanically deformable than said member distal end section;
whereby having said member distal end section substantially more mechanically deformable than said member middle section reduces risks of injuring said tissue with said member distal end section when positioning said member distal end section substantially adjacent said aperture while allowing for the transmission of substantially longitudinal forces from said member proximal end section to said member distal end section to enlarge said aperture by pushing, at least in part, said member distal end section through said aperture.

2. A dilator as defined in claim 1, wherein said reinforcing component is substantially tubular and extends substantially longitudinally within said tube lumen, and wherein said reinforcing component and said tube lumen are substantially coaxial.

3. A dilator as defined in claim 1, wherein said member distal end section extends substantially longitudinally from said tube.

4. A dilator as defined in claim 3, wherein said member distal end section is tapered in a direction leading substantially away from said member middle section.

5. A dilator as defined in claim 1, wherein a ratio between a length of said member distal end section and a length of said member middle section is from about 1:200 to about 1:33.

6. A dilator as defined in claim 1, wherein said reinforcing component includes a metal and wherein said component distal end section is substantially electrically insulating.

7. A dilator as defined in claim 1, wherein said reinforcing component is made out of annealed stainless steel.

8. A dilator as defined in claim 1, wherein said member distal end section is made out of a material selected from the group consisting of polyetheretherketone, polyethylene block amide, polyurethane, and polyvinylchloride.

9. A dilator as defined in claim 1, wherein said reinforcing member defines a reinforcing member radially outermost surface and said tube defines a tube radially innermost surface, said reinforcing member radially outermost surface and said tube radially innermost surface being in contact with each other.

10. A dilator as defined in claim 9, wherein said reinforcing member radially outermost surface and said tube radially innermost surface are bonded to each other.

11. A dilator as defined in claim 1, wherein said reinforcing component defines a reinforcing component proximal end and a substantially opposed reinforcing component distal end, said reinforcing component proximal end being located outside of said member middle section.

12. A dilator as defined in claim 11, wherein said reinforcing component is flared substantially adjacent said member proximal end section.

13. A dilator as defined in claim 1, wherein a flexural rigidity of said member middle section is at least about 30 times larger than a flexural rigidity of said member distal end section.

14. A dilator as defined in claim 1, wherein a flexural rigidity of said member middle section is at least about 0.05 N/m².

15. A dilator as defined in claim 1, wherein said substantially elongated member includes a curved section and wherein said curved section traverses from about 30° to about 89° of a circle.

16. A dilator as defined in claim 1, wherein said substantially elongated member is substantially tubular, said substantially elongated member having an inner diameter of from about 0.08 cm to about 0.18 cm and an outer diameter of from about 0.15 cm to about 0.37 cm.

17. A dilator as defined in claim 16, wherein said substantially elongated member has an inner diameter of about 0.15 cm and an outer diameter of about 0.25 cm.

18. A dilator as defined in claim 1, wherein said substantially elongated member has a length of from about 50 cm to about 100 cm.

19. A dilator as defined in claim 1, wherein said member distal end section has an end section length of from about 5 mm to about 15 mm.

20. A dilator as defined in claim 1, further comprising a radiopaque marker coupled to said member distal end section.

21. A dilator as defined in claim 1, wherein said member distal end section defines a distal end section lumen extending substantially longitudinally therethrough, said distal end section lumen being in fluid communication with said tube lumen, said distal end section further defining a lateral aperture extending substantially radially outwardly from said distal end section lumen.

22. A dilator as defined in claim 1, wherein said reinforcing member defines a reinforcing member radially outermost surface, said reinforcing member outermost surface being, at least in part, substantially roughened.

23. A dilator, said dilator being positionable substantially adjacent an aperture in a tissue and usable to enlarge said aperture, said dilator comprising:
a substantially elongated member, said substantially elongated member defining a member proximal end section, a substantially longitudinally opposed member distal end section and a member middle section extending therebetween, said member middle section being substantially less mechanically deformable than said component distal end section;
whereby having said member distal end section substantially more mechanically deformable than said member middle section reduces risks of injuring said tissue with said member distal end section when positioning said member distal end section substantially adjacent said aperture while allowing for the transmission of substantially longitudinal forces from said member proximal end section to said member distal end section to enlarge said aperture by pushing, at least in part, said member distal end section through said aperture.
